(19) United States
(12) Patent Application Publication
(10) Pub. No.: US 2009/0292175 A1
(43) Pub. Date: Nov. 26, 2009

(54) MEDICAL DEVICE

(75) Inventors: Shunya AKIMOTO, Kawasaki-shi (JP); Junichi ONISHI, Tokyo (JP); Seiichi ITO, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/468,277

(22) Filed: May 19, 2009

(30) Foreign Application Priority Data
May 23, 2008 (JP) 2008-135635

(51) Int. Cl. A61B 1/267; G06K 9/00

(52) U.S. Cl. 600/156; 600/160; 600/153; 382/128

(57) ABSTRACT

A medical device for examination or treatment based on a reference point, including: an image pickup section capable of picking up an image of a bronchus in a patient; a VBS image generation section configured to generate a virtual endoscopic image of bronchus in a patient from a plurality of different line-of-sight positions based on three-dimensional image data of the patient that is obtained in advance; an image retrieval section configured to retrieve a virtual endoscopic image highly similar to the endoscopic image of the bronchus picked up by the image pickup section; and a reference-point setting section configured to set a predetermined position near the image pickup section as a reference point based on the line-of-sight positions of the highly similar virtual endoscopic image.

This application claims the benefit of Japanese Application No. 2008-135635 filed in Japan on May 23, 2008, the contents of which are incorporated herein by this reference.

### BACKGROUND OF THE INVENTION

1. Field of the Invention

The present invention relates to a medical device having an image pickup section that is able to pick up images of a tube cavity in a subject, more particularly, to a medical device that performs examination or treatment with high accuracy using virtual endoscopic images of a tube cavity based on three-dimensional image data of a subject.

2. Description of the Related Art

In recent years, diagnoses have been widely made using three-dimensional images. For example, a diagnosis for a target site can be made using three-dimensional image data of a subject which is obtained by picking up tomographic images of the subject with an X-ray CT (Computed Tomography) apparatus.

In the CT apparatus, a subject is continuously moved while X-ray radiating positions and detection positions are continuously rotated for continuous helical scanning of the subject (helical scan). A number of the resulting continuous two-dimensional tomographic images of the subject are used to create a three-dimensional image.

A three-dimensional image of bronchus of lungs is one type of the three-dimensional images used in diagnoses. Such a three-dimensional image of bronchus is used in a three-dimensional detection of the position of a diseased area with suspected lung cancer for example. In order to check the diseased area by a biopsy, a bronchus endoscope is inserted into the subject and a biopsy needle or biopsy forceps are extended out from a distal end portion of the bronchus endoscope, so as to collect tissue samples of the target site.

In a tract such as bronchus in a body that is branched in multiple steps, in a case where a diseased area is located at the end of a bronchus, it is hard to bring the distal end of an insertion section of an endoscope to a position near the target site in a short period of time with accuracy. Thus, for example, Japanese Patent Application Laid-Open Publication No. 2004-180940 and Japanese Patent Application Laid-Open Publication No. 2005-131042 disclose navigation systems for insertion of endoscope in which a three-dimensional image of a tube cavity in a subject is created based on image data of a three-dimensional area in the subject, and a route along the tract to a target on the three-dimensional image is obtained, so that virtual endoscopic images of the tract along the route can be created based on the image data.

### SUMMARY OF THE INVENTION

A medical device of the present invention includes: an image pickup section that is able to pick up an image of a tube cavity in a body of a subject; a virtual endoscopic image generation section configured to generate a virtual endoscopic image in the tube cavity from a plurality of different line-of-sight positions based on three-dimensional image data of the subject that is obtained in advance; an image retrieval section configured to retrieve a virtual endoscopic image highly similar to the endoscopic image of the tube cavity picked up by the image pickup section; and a reference-point setting section configured to set a predetermined position near the image pickup section as a reference point based on the line-of-sight positions of the highly similar virtual endoscopic image.

### BRIEF DESCRIPTION OF THE DRAWINGS

FIG. 1 is a configuration view showing a configuration of a medical device of an embodiment according to the present invention;

FIG. 2 is an illustrative view of a use form of the medical device of the embodiment according to the present invention;

FIG. 3 is a view showing an example of a screen display of navigation in inserting an endoscope which is the medical device of the embodiment according to the present invention to a position near a target site of a tube cavity in a subject body;

FIG. 4 is a view showing an example of a format of a screen display while an insertion navigation system of the medical device of the embodiment according to the present invention is being operated;

FIG. 5 is a flow chart illustrating a process flow for calculating a position and a direction of an end of the medical device of the embodiment according to the present invention;

FIG. 6 is a view showing an example of the relationship between the number of times loop operations that are repeated and an error in the medical device of the embodiment according to the present invention;

FIG. 7A is a schematic front view illustrating a configuration of a distal end portion of the medical device of the embodiment according to the present invention;

FIG. 7B is a schematic cross-sectional view taken along the VII-B-VII-B line of FIG. 7A;

FIG. 8 is a schematic perspective view illustrating a distal end portion of the medical device of the embodiment according to the present invention;

FIG. 9A is an illustrative view illustrating a setting of a reference point by the medical device of the embodiment according to the present invention; and

FIG. 9B is an illustrative view illustrating a setting of a reference point by the medical device of the embodiment according to the present invention.

### DETAILED DESCRIPTION OF THE PREFERRED EMBODIMENTS

Now, with reference to the drawings, an embodiment according to the present invention will be explained below.

### <Description of Device Configuration>

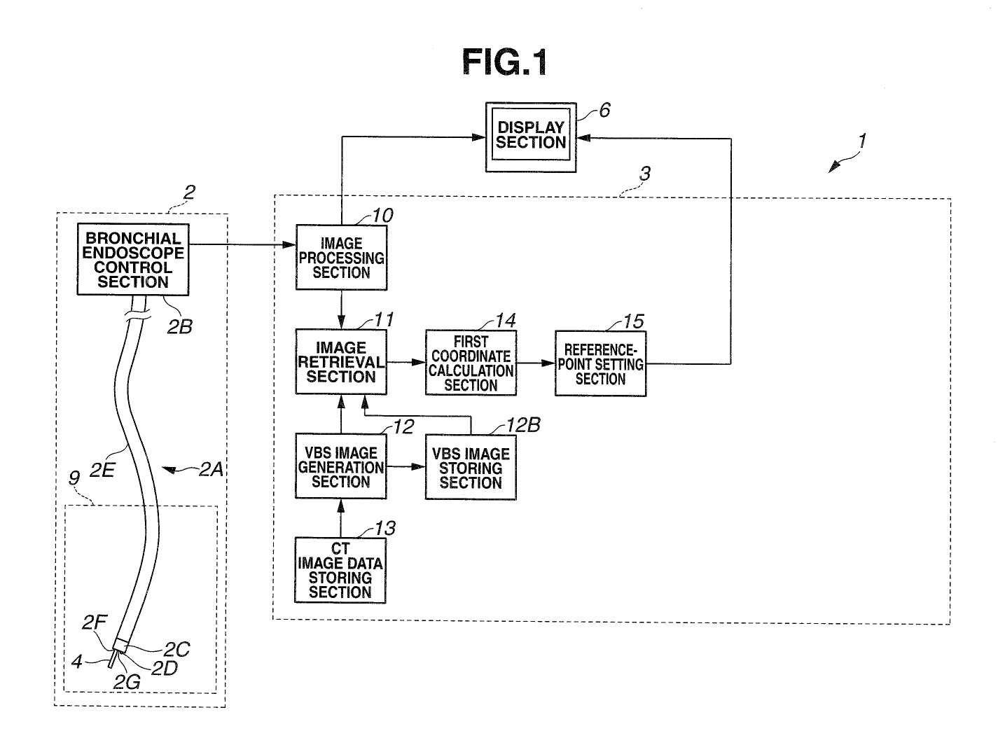

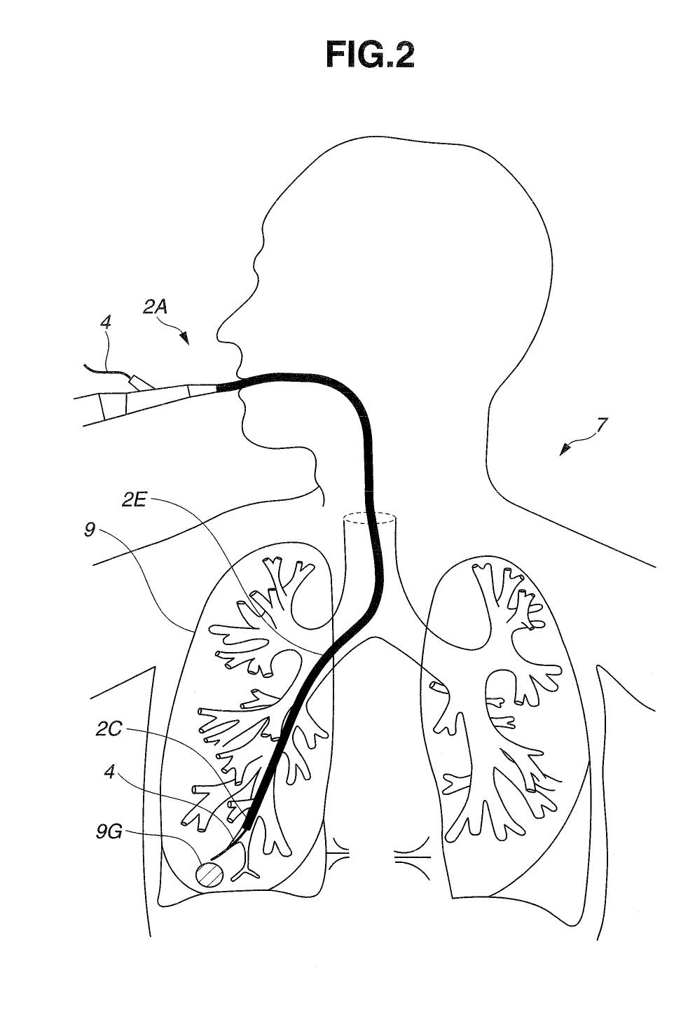

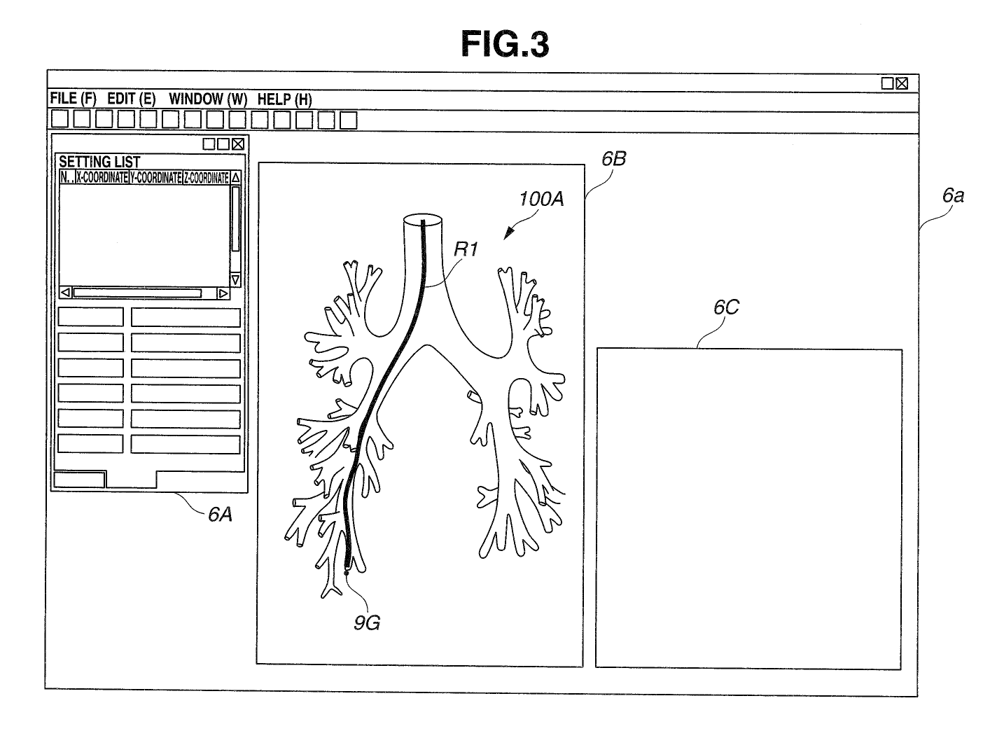

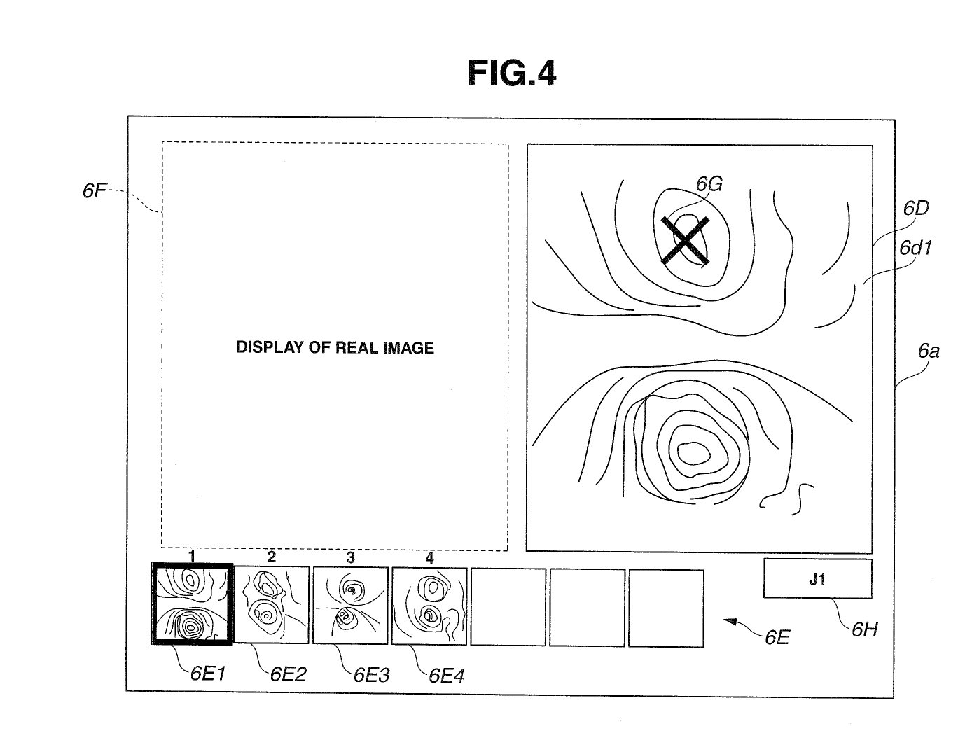

FIG. 1 is a configuration view showing a configuration of a medical device 1 of the embodiment according to the present invention; FIG. 2 is an illustrative view of a use form of the medical device 1; and FIGS. 3 and 4 are views showing examples of a screen display of an insertion navigation in inserting a distal end portion 2C of an endoscope 2A to a position near a target site 9G of a bronchus of a patient 7.

As shown in FIG. 1, the medical device 1 of the present embodiment includes: an endoscope apparatus 2 having the endoscope 2A configured to be inserted to the bronchus 9 that is a tube cavity in a body of the patient 7 who is the subject to pick up images of the inside of the bronchus 9 and to perform a biopsy of the target site 9G at the end of the bronchus 9 (see FIG. 2); and an insertion assist apparatus 3.

The endoscope apparatus 2 includes: an insertion section 2E insertable through the bronchus 9 of the patient 7; an endoscope 2A having an image pickup section 2D such as a CCD arranged at a distal end portion 2C of the elongated insertion section 2E; an endoscope control section 2B configured to control the endoscope 2A; a display section 6; and the like. The display section 6 also displays information of an insertion assist apparatus 3. The insertion section 2E has a channel (not shown) formed therein through which the treatment instrument 4 as a medical instrument can be inserted, and the distal end portion 2C is provided with a treatment instrument port 2F that is an opening of a channel 2F1, thereby as shown in FIG. 1, the treatment instrument 4 can be protruded from the treatment instrument port 2F.

The insertion section 2E also has a tract (not shown) formed therein for liquid-supply, and the distal end portion 2C is provided with a liquid-supply port 2G as an opening of the tract. Therefore, a surgeon is able to eject or spray a desired liquid from the liquid-supply port 2G to the target site 9G.

FIG. 2 shows a states where the distal end portion 2C of the insertion section 2E is inserted into a tube cavity of the bronchus 9 of the patient 7 having the minimal diameter for insertion of the distal end portion 2C, and the treatment instrument 4 that is a medical instrument having a small diameter is protruded out of the distal end portion 2C, and samples the tissues of the target site 9G.

As shown in FIG. 2, the insertion section 2E of the endoscope apparatus 2 is narrow so as to be insertable into narrow bronchus tube cavities, and has a diameter on the order of 3 mm for example, but the treatment instrument 4 has a diameter on the order of 1 mm so as to be insertable into narrower end parts of the bronchus tube cavities. In many cases, the target site 9G at an end of a bronchus cannot be checked due to the narrowness by an image pickup section 2D that is arranged at the distal end portion 2C.

The insertion assist apparatus 3 includes: an image processing section 10; a CT image data storing section 13; a virtual endoscopic image (Virtual Bronchus Scope Image: hereinafter, also referred to as “VBS image”) generation section 12 configured to generate a virtual endoscopic image; an image retrieval section 11 configured to retrieve a virtual endoscopic image highly similar to an endoscopic image; and a first coordinate calculation section 14. The image processing section 10 processes an endoscopic image (hereinafter, also referred to as “real image”) picked up by the image pickup section 2D. The CT image data storing section 13 stores three-dimensional image data in a format such as DICOM (Digital Imaging and Communication in Medicine) that is generated by a known CT apparatus (not shown) for picking up X-ray tomographic images of the patient 7. The VBS image generation section 12 generates a VBS image from the image data in DICOM format based on line-of-sight parameters which will be explained later.

The insertion assist apparatus 3 may further include a VBS image storing section 12B configured to store the VBS images generated by the VBS image generation section 12, and a reference point setting section 15 configured to calculate a reference point based on a first coordinate. The image retrieval section 11 and the first coordinate calculation section 14 will be explained later in detail.

First, with reference to FIGS. 3 and 4, an insertion navigation system will be simply explained below. FIG. 3 is a view showing an example of a screen display of a display section 6 that is displaying an insertion route and the like for inserting the insertion section 2E to the bronchus 9; and FIG. 4 is a view showing an example of a format of a screen display while an insertion navigation system is being operated. However, the medical device 1 of the present embodiment does not necessarily have to be provided with a function of insertion navigation system.

As shown in FIG. 3, when the insertion navigation system implements an insertion navigation, first, the display section 6 displays: information of the patient 7; information 6A of branches J of the bronchus 9; an image 6B displaying an insertion route R1 of the endoscope 2A; a VBS image 6C (not shown in detail), and the like, on the screen display 6a. The image 6B shows the insertion route R1 of the endoscope 2A to the target site 9G which is set by the insertion navigation system so that the insertion route R1 is superimposed on a bronchus image 100A of the patient 7 generated based on a three-dimensional image.

Upon a start of an insertion operation of the endoscope, next, as shown in FIG. 4, the display section 6 displays: an endoscope real image display area 6F for displaying a real image picked up by the image pickup section 2D and processed by the image processing section 10; a VBS image display area 6D for displaying a VBS image 6d; a display area 6H for displaying a name of the branch displayed in the VBS image 6d, an order of the branch, and the like; and branch-thumbnail VBS images 6E for displaying size-reduced VBS images at all of the branches along an insertion route to a target site as branch-thumbnail VBS images, on the screen display 6a.

FIG. 4 shows an insertion navigation screen for a route to a target site via four branches J1, J11, J111, and J1111. That is, the VBS image display area 6D displays a VBS image 6d1 of the first branch J1 along the route, and the branch-thumbnail VBS images 6E include branch-thumbnail VBS images 6E1 to 6E4 at all of the above four branches along the insertion route. On the VBS image 6d1, a marker 6G is superimposed at a route hole which leads an insertion route 1.

When a surgeon inserts the distal end portion 2C into a route hole having the marker 6G superimposed thereon, the VBS image 6d on the insertion navigation screen is switched to the VBS image at the second branch.

As a result, using the insertion navigation system, the surgeon is able to insert the distal end portion 2C to the branch of the bronchus 9 having the minimal diameter for insertion of the distal end portion 2C to a position near the target site 9G without making a mistake, at every branch, in determining a route hole in which the distal end portion 2C should be inserted.

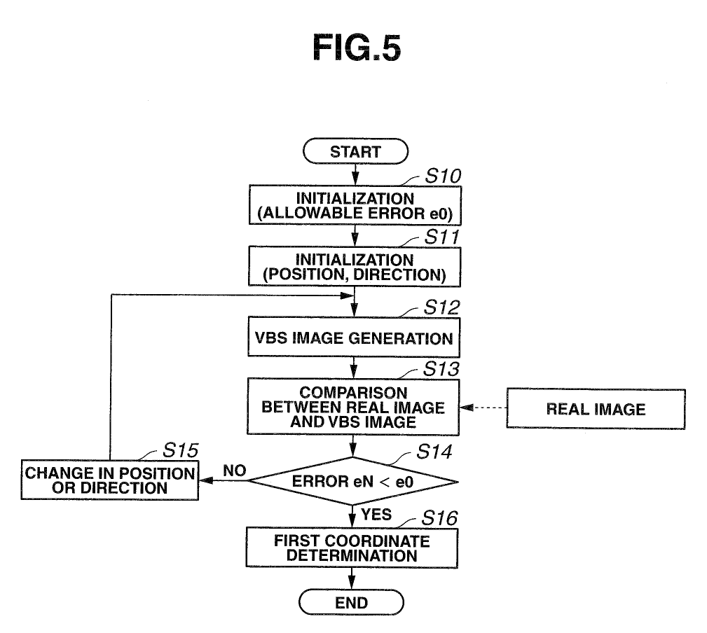

After the distal end portion 2C is inserted to a position near the target site 9G, in the medical device 1, the first coordinate calculation section 14 calculates a position (a three-dimensional coordinate) and a direction of the distal end portion 2C. FIG. 5 is a flow chart illustrating a process flow for calculating a position and a direction of the distal end portion 2C in the medical device 1. Now, in accordance with the flowchart of FIG. 5, a process flow for calculating a position and a direction of an endoscope distal end portion by the medical device 1 will be explained below.

### <Step S10>

First, an allowable error e0 is set for determination of similarity which is performed by the image retrieval section 11. A smaller allowable error e0 allows the first coordinate calculation section 14 to more accurately calculate the position and direction of the distal end portion 2C, but takes a longer time. Thus, the allowable error e0 is changeable by a surgeon depending on the purpose.

### <Step S 11>

The VBS image generation section 12 can generate a VBS image from a large number of different line-of-sight positions based on image data in DICOM format by changing six line-of-sight parameters. The parameters of line-of-sight positions as used herein are six-dimensional parameters including positions (x, y, z) and angles (θx, θy, θz). At Step S11, the six initial values of the line-of-sight parameters are set.

In a case where the endoscope 2A is inserted using the insertion assist apparatus 3 that is provided with a function of insertion navigation system, the line-of-sight parameters of the VBS image at the last branch J are preferably set as the initial values.

### <Step S12>

The VBS image generation section 12 generates one VBS image using three-dimensional image data of the bronchus 9 of the patient 7 stored in the CT image data storing section 13, based on the initial values of the line-of-sight parameters.

<Step S13>The image retrieval section 11 compares the real image and the VBS image generated by the VBS image generation section 12 on the similarity to each other. The comparison between the images is performed by a known image process which may be matching process on the level of pixel data or matching process on the level of features extracted from images. Because the matching process between the real image and the VBS image is performed for every frame of the real image, the actual comparison is made based on the similarity between the static endoscopic image and the virtual endoscopic image. The matching process need not be performed for all of the frames of the real image, but is performed at appropriate intervals.

### <Step S14 and Step S15>

When the error e calculated by the image retrieval section 11 for the similarity between the real image and the VBS image is larger than the allowable error e0 (No), at Step S15, the image retrieval section 11 outputs a line-of-sight parameter of a slightly different line-of-sight position or direction to the VBS image generation section 12. Then, at Step S12, the VBS image generation section 12 generates a next VBS image according to the new line-of-sight parameter set at Step S15.

The insertion assist apparatus 3 repeats the above loop operations, that is, outputs a different line-of-sight parameters, and as a result of that the VBS image generated by the VBS image generation section 12 is gradually changed to an image similar to the real image, and the error e between the images becomes equal to the allowable error e0 or less (Yes) after the loop operations are repeated some times.

### <Step S16>

When the similarity error e between the VBS image and the real image becomes equal to the allowable error e0 or less, the first coordinate calculation section 14 calculates a position (coordinate) and a direction of the distal end portion 2C using the line-of-sight parameters of the VBS image having higher similarity.

As explained at Step S13, the real image is updated at appropriate intervals, but while the distal end portion 2C remains at a position, because the position and the direction of the distal end portion 2C do not significantly change, the number of times the loop operations are repeated is not increased in spite of the updates of the real image. However, a large movement of the distal end portion 2C causes a large change in the real image, which requires a large number of repetitions of the above described loop operations to make the errors e of the images equal to an allowable error e0 or less. During a biopsy of the target site 9G, because the distal end portion 2C does not usually move a lot, any large movement of the distal end portion 2C implies an abnormal situation. So, when the medical device 1 determines that there is a large change in a real image, that is, the error e of the loop operations is suddenly increased, the medical device 1 preferably generates an alarm, and discontinues the loop operations for a predetermined period of time, for example, several seconds.

In the above description, the endoscope 2A is operated to be inserted by the insertion assist apparatus having a function of insertion navigation system, but even when the endoscope 2A is operated to be inserted without the insertion navigation system, the first coordinate calculation section 14 of the medical device 1 is able to calculate a position and a direction of the distal end portion 2C.

For example, the initial values for a line-of-sight position used at Step S11 may be randomly determined. Alternatively, the VBS image generation section 12 may generate virtual endoscopic images at branches of the bronchus in advance from a plurality of different line-of-sight positions, and store the image in the VBS image storing section 12B, so that the image retrieval section 11 can retrieve the VBS image most highly similar to a real image from the stored VBS images, and set the line-of-sight parameters of the most highly similar VBS image as initial values at the line-of-sight position that are used at Step S11.

Also, in the above description, a position and a direction of the distal end portion 2C is calculated using the six-dimensional line-of-sight parameters by the first coordinate calculation section 14 when the error e of the similarity between the VBS image and the real image becomes equal to the allowable error e0 or less. However, the first coordinate calculation section 14 may calculate a position and a direction of the distal end portion 2C using the line-of-sight parameters of a VBS image which is determined to be most highly similar to the real image by the image retrieval section 11 among the virtual endoscopic images of bronchus branches from a plurality of different line-of-sight positions generated by the VBS image generation section 12 in advance. Needless to say, in the latter case, the number of VBS images generated in advance should be increased to enhance the accuracy of the obtained position and direction of the distal end portion 2C.

As explained above, the VBS image generation section 12 generates a plurality of VBS images based on different line-of-sight parameters using the information from the image retrieval section 11 so as to generate a more highly similar VBS image, that is, a VBS image having a smaller error e. Generally, the VBS images generated by the VBS image generation section 12 at early steps of the above loop operations have larger errors e, but the more VBS images are generated, the smaller error e becomes.

As already explained above, the image retrieval section 11 compares the real image and the VBS image generated based on line-of-sight parameters by the VBS image generation section 12 on the similarity, and when the error e of the similarity between the images becomes equal to the allowable error e0 or less, the first coordinate calculation section 14 sets a coordinate of the line-of-sight positions of the VBS image as a first coordinate.

In the medical device 1, preferably, the allowable error e0 is individually set to be different in a tube cavity longitudinal direction ZA and in a tube cavity orthogonal direction YA. The tube cavity longitudinal direction ZA as used herein is the longitudinal direction of a bronchus tube cavity through which the distal end portion 2C is inserted and along which the distal end portion 2C moves in accordance with an insertion operation. When VBS image data includes information of the centroidal line of a tube cavity, so-called centerline information, the allowable error e0 may be changed by using the centerline direction instead of the longitudinal direction of the tube cavity, and the direction orthogonal to the centerline direction instead of the tube cavity orthogonal direction.

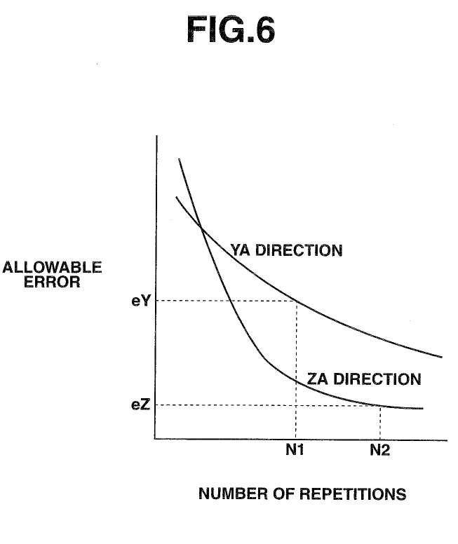

FIG. 6 is a view showing an example of the relationship between the number of VBS images generated by the VBS image generation section 12, in other words, the number of times N the loop operations are repeated and an error e. As shown in FIG. 6, the error e of a VBS image which is generated by changing the coordinate of the tube cavity longitudinal direction ZA or the coordinate of the tube cavity orthogonal direction YA as a line-of-sight parameter is decreased as the number of times N is increased. Herein, in the medical device 1, an allowable error eZ is set for a VBS image that is generated by changing only the coordinate of the tube cavity longitudinal direction ZA as a parameter, and an allowable error eY is set for a VBS image that is generated by changing only the coordinate of the tube cavity orthogonal direction YA as a parameter, so that the allowable error eZ is smaller than the allowable error eY. That is, in the medical device 1, preferably the accuracy of the coordinate of the tube cavity longitudinal direction ZA is valued more than the accuracy of the coordinate of the tube cavity orthogonal direction YA in the positional information of a calculated first coordinate point.

Alternatively, in the medical device 1, the accuracy of the coordinate of the tube cavity longitudinal direction ZA can be higher than the accuracy of the coordinate of the tube cavity orthogonal direction YA in the positional information of a calculated first coordinate point, by setting an increment/decrement of the coordinate of the tube cavity longitudinal direction ZA to be smaller than an increment/decrement of the coordinate of the tube cavity orthogonal direction YA at Step S15 during the loop operations shown in FIG. 5.

This is because the distal end portion 2C in a narrow tube cavity having a diameter similar to the diameter of the distal end portion 2C hardly moves in the tube cavity orthogonal direction YA in case of any wrong operation, but easily moves in the tube cavity longitudinal direction ZA. Also, this is because a treatment is performed with the treatment instrument 4 being protruded from the treatment instrument port 2F of the channel 2F1, based on the coordinate of the tube cavity longitudinal direction ZA.

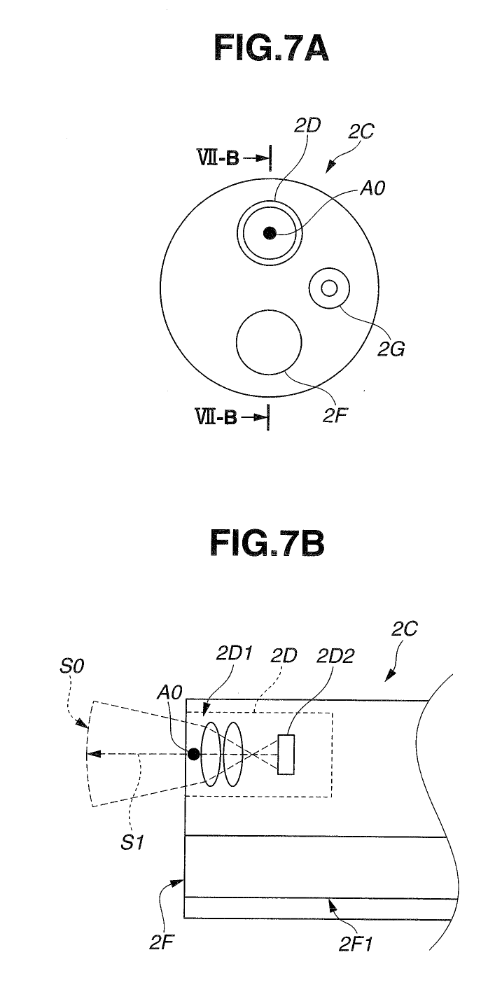

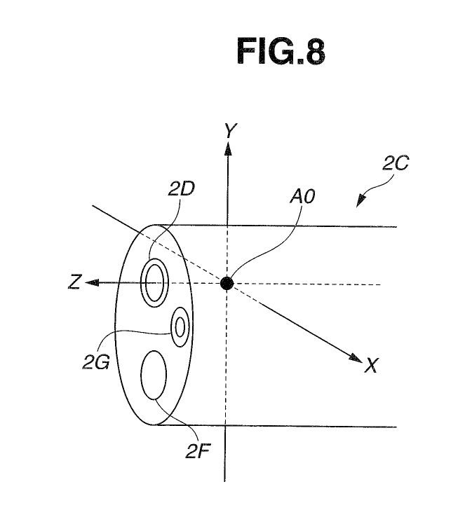

Now, with FIG. 7A, FIG. 7B, and FIG. 8, a structure of the distal end portion 2C will be explained below in more detail. FIG. 7A is a schematic front view illustrating a configuration of the distal end portion 2C; FIG. 7B is a schematic cross-sectional view taken along the VII-B-VII-B line of FIG. 7A; and FIG. 8 is a perspective schematic view of the distal end portion 2C.

As shown in FIG. 7A, FIG. 7B, and FIG. 8, the distal end portion 2C is provided with the treatment instrument port 2F that is an opening of the channel 2F1, the a liquid-supply port 2G that is an opening of the liquid-supply tract, and the image pickup section 2D. The distal end portion 2C is further provided with an illumination section configured to illuminate the inside of a tube cavity, and the like, which are not shown. The image pickup section 2D has an image pickup device 2D2 therein at the focus position of an optical system 2D1 to pick up an image within a field of view S0 in the direction with a line-of-sight S1 as a center.

The point on endoscope that corresponds to a line-of-sight parameter of a VBS image represented by a first coordinate point calculated by the first coordinate calculation section 14 constitutes a pupil position A0 and the direction of the line-of-sight S1 as often called in an optical system.

Here, the coordinate of the first coordinate point A0 is expressed in a coordinate system of the virtual endoscopic image, in other words, a CT coordinate system, which means a lot to the medical device 1. That is, as already explained above, because the target site 9G for a biopsy and the like is located at a bronchus end which the distal end portion 2C cannot reach, a surgeon cannot perform a biopsy and the like using the treatment instrument 4 while checking real images for the target site 9G. Therefore, a surgeon performs a biopsy based on the position of the target site 9G shown in a CT coordinate system using the display section 6 in the three-dimensional image data that is obtained by a CT apparatus in advance. However, the surgeon can check the position of the distal end portion 2C, more specifically, the position of the distal end of the treatment instrument 4 for the biopsy protruded from the distal end portion 2C only in an endoscope coordinate system based on the distal end portion 2C which has no relationship with the CT coordinate system.

To the contrary, in the medical device 1, the coordinate of the first coordinate point A0 on a part of the distal end portion 2C that is close to the coordinate of the target site 9G is expressed in the same CT coordinate system, which allows the surgeon to use the coordinate to bring the treatment instrument 4 to the target site 9G for a biopsy and the like based on the first coordinate point A0 as a reference point.

The examination or treatment performed using the medical device 1 herein may be spray of medication, biopsy, mucus sampling, extraction of foreign object, high-frequency cauterization, or the like. And, the treatment instrument 4 such as forceps or a probe such as an ultrasound probe as a medical instrument depending on the examination or treatment is inserted through the channel 2F1 for use.

The endoscope coordinate system for the medical device 1 shown in FIG. 8 is not the same with the CT coordinate system, but is a coordinate system which is processed to correspond to the CT coordinate system by the insertion assist apparatus 3, in other words, a coordinate system which can be transformed into the CT coordinate system by a coordinate transformation process.

In the above description, the first coordinate point A0 is used as a reference point as it is, but in the medical device 1, based on the first coordinate point A0, another position represented in an endoscope coordinate system which is set by the reference-point setting section 15 using a transformation parameter may be set as a reference point.

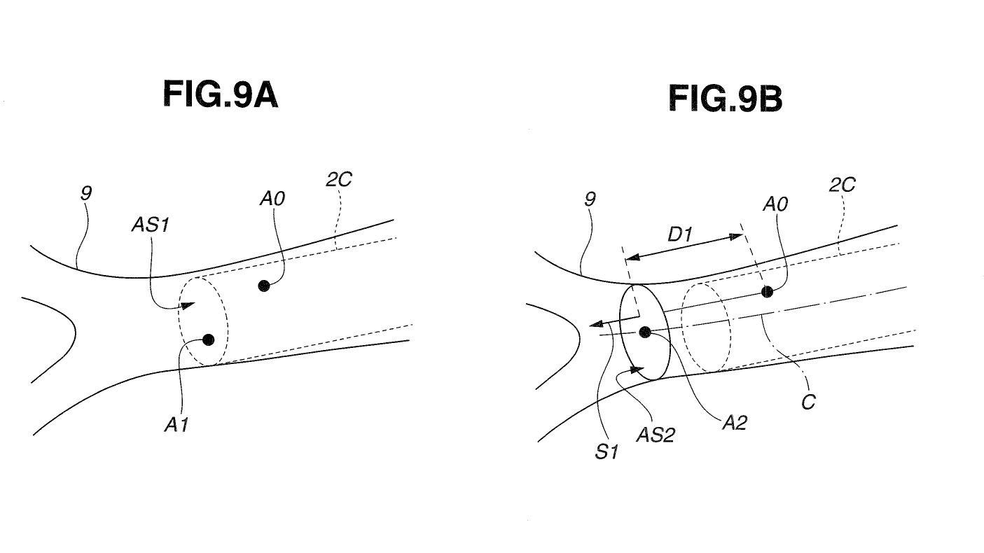

FIGS. 9A and 9B are illustrative views illustrating settings of a reference point.

FIG. 9A shows a method for setting a reference point, in which the reference point A1 is set on the distal end plane AS1 of the distal end portion 2C. The distal end plane AS1 means a flat plane that includes a predetermined position on the distal end portion 2C, and does not mean that the distal end of the insertion section 2E is provided with a flat plane.

FIG. 9B shows a method for setting a reference point, in which a reference point A2 is set on a plane having a predetermined relationship with a line-of-sight position of a virtual endoscopic image highly similar to a real image, in other words, in an area of a plane having a predetermined relationship that is determined based on a line-of-sight position in advance. That is, in FIG. 9B, an example is shown, in which a reference point A2 is set on the plane AS2 that is separated by a predetermined distance D1 in the direction of a line-of-sight S1 and is orthogonal to the direction of the line-of-sight S1, based on the first coordinate point A0 that is a line-of-sight position. In FIG. 9B, the plane AS2 is illustrated as a plane orthogonal to the direction of the line-of-sight S1, but may be a flat plane that is not orthogonal but inclines at an angle, or a curved plane having a predetermined shape, depending on the shape of an endoscope.

As the reference point Al, for example, a position in the liquid-supply port 2G or the treatment instrument port 2F, more specifically, the center of the liquid-supply port 2G or the treatment instrument port 2F is used. That is, the reference point A1 is set at the center of the liquid-supply port 2G or the treatment instrument port 2F. Also, as the reference point A2, an intersection point of the central line of the tube cavity, for example, the centerline C1 and the plane AS1 is selected.

As compared to the case where the first coordinate point A0 is used as a reference point, the above described reference point A1 enables more precise examination or treatment because the liquid-supply port 2G for treatment and the like or the treatment instrument port 2F providing a starting point from which the treatment instrument 4 is protruded is used as a reference point. In addition, the above described reference point A2 facilitates the calculation of a distance to the target site 9G.

As explained above, the medical device 1 for examination or treatment based on a reference point sets a predetermined position at a part near the image pickup section 2D which is located near the target site 9G as a reference point. The position at a part near the image pickup section 2D is in the bronchus that is a tube cavity in a body of the patient 7 who is the subject, and is a predetermined position in the medical device 1 including the inside of the image pickup section 2D. The position near the image pickup section 2D is preferably in the bronchus between a line-of-sight position of the image pickup section 2D and the target site 9G, more preferably a predetermined position on the distal end portion 2C.

In the above description, only one reference point is set, but a plurality of reference points may be set, and alternatively, a reference plane may be set instead of a reference point.

As explained above, a medical device of the present invention includes:

a treatment instrument or probe for examination or treatment based on a reference point;

an insertion section having a channel formed therein through which the treatment instrument or the probe is insertable, and having an opening of the channel and an image pickup section that is able to pick up an image of bronchus of a subject at a distal end portion thereof;

a virtual endoscopic image generation section configured to generate a virtual endoscopic image in the bronchus from a plurality of line-of-sight positions based on three-dimensional image data of the subject that is obtained in advance, and also generate a more highly similar virtual endoscopic image based on information of a most highly similar virtual endoscopic image retrieved by an image retrieval section configured to retrieve the virtual endoscopic image most highly similar to the endoscopic image of the bronchus picked up by the image pickup section among the plurality of the virtual endoscopic images that are generated in advance; and

a reference-point setting section configured to set a position in the opening as a reference point based on the line-of-sight positions of the more highly similar virtual endoscopic image.

In the above description, the example with the endoscope apparatus 2 having the elongated insertion section 2E is used, but a medical device of the present invention may be a medical device such as a capsule endoscope apparatus having an image pickup section 2D that is able to pick up an image of tube cavity in the body of a patient 7, which also provides the same operational effects as those of the endoscope apparatus 2 having the elongated insertion section 2E.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A medical device, comprising:
an image pickup section that is able to pick up an image of a tube cavity in a body of a subject;
a virtual endoscopic image generation section configured to generate a virtual endoscopic image in the tube cavity from a plurality of different line-of-sight positions based on three-dimensional image data of the subject that is obtained in advance;
an image retrieval section configured to retrieve a virtual endoscopic image highly similar to the endoscopic image of the tube cavity picked up by the image pickup section; and
a reference-point setting section configured to set a predetermined position near the image pickup section as a reference point based on the line-of-sight positions of the highly similar virtual endoscopic image.

2. The medical device according to claim 1, wherein
the virtual endoscopic image generation section generates a more highly similar virtual endoscopic image based on information from the image retrieval section, and
the reference-point setting section sets the reference point based on the line-of-sight position of the more highly similar virtual endoscopic image generated by the virtual endoscopic image generation section.

3. The medical device according to claim 1, wherein
the image retrieval section retrieves the most highly similar virtual endoscopic image among the plurality of the virtual endoscopic images generated by the virtual endoscopic image generation section in advance; and
the virtual endoscopic image generation section generates a still more highly similar virtual endoscopic image based on the information of the most highly similar virtual endoscopic image retrieved by the image retrieval section, and
the reference-point setting section sets the reference point based on the line-of-sight positions of the still more highly similar virtual endoscopic image generated by the virtual endoscopic image generation section.

4. The medical device according to claim 1, wherein
the virtual endoscopic image generation section generates the virtual endoscopic image having a coordinate in the tube cavity longitudinal direction and a coordinate in the tube cavity direction orthogonal to the tube cavity longitudinal direction that is different from the coordinate of the tube cavity longitudinal direction, and
the image retrieval section retrieves the more highly similar virtual endoscopic image based on the similarity between the virtual endoscopic images having different coordinates in the tube cavity longitudinal direction rather than the similarity between the virtual endoscopic images having different coordinates in the tube cavity orthogonal direction.

5. The medical device according to claim 1, wherein
the reference point is set in a region on a plane having a predetermined relationship with a line-of-sight position of the highly similar virtual endoscopic image.

6. The medical device according to claim 1, further comprising:
an insertion section that is insertable into the tube cavity, has a channel formed therein through which a treatment instrument or a probe is insertable, and is provided with an opening of the channel and the image pickup section at a distal end portion thereof, and
the reference point is set in the opening.

7. The medical device according to claim 1, further comprising:
an insertion section that is insertable into the tube cavity, has a tract formed therein for liquid-supply, and is provided with a liquid-supply port of the tract at a distal end portion thereof, and
the reference point is set in the liquid-supply port.

8. The medical device according to claim 1, which performs examination or treatment based on the reference point.

9. The medical device according to claim 1, wherein
the tube cavity is bronchus.
